(19) United States
(12) Patent Application Publication
(10) Pub. No.: US 2026/0207163 A1
(43) Pub. Date: Jul. 23, 2026

(54) METHOD, DEVICE AND DEVICE ARRANGEMENT FOR CLASSIFYING A CEREBRAL PRESSURE, AND CORRESPONDING COMPUTER PROGRAM AND COMPUTER-READABLE MEDIUM

(71) Applicant: Sonovum GmbH, Leipzig (DE)

(72) Inventors: Bertram König, Dresden (DE); Raiko Klemp, Leipzig (DE); Florian Tetschke, Dresden (DE)

(21) Appl. No.: 19/142,450

(22) PCT Filed: Dec. 19, 2023
(86) PCT No.: PCT/EP2023/086588

§ 371 Date: Jun. 23, 2025

(30) Foreign Application Priority Data
Dec. 30, 2022 (DE) 10 2022 135 061.4

(51) Int. Cl. A61B 8/08; A61B 5/03; A61B 8/00

(52) U.S. Cl. CPC A61B 8/0808; A61B 5/031; A61B 8/5223; A61B 8/5269; A61B 8/58

(57) ABSTRACT

A computer-implemented method for classifying a brain pressure, the method comprising: receiving data comprising time-of-flight values, which are based on time-of-flight values measured at successive time points for one or more ultrasound signals through a cross-section of a skull; classifying a characteristic of the brain pressure by means of one or more features of a representative curve of a time course of the time-of-flight values to obtain a classified characteristic of the brain pressure; and outputting the classified characteristic of the brain pressure.

The invention relates to a computer-implemented method for classifying a brain pressure, an apparatus for classifying the brain pressure, an apparatus arrangement for classifying the brain pressure and a corresponding computer program and a computer-readable medium.

The brain is constantly supplied with oxygen and nutrients via the blood. If the blood flow through the brain is reduced, damage to the brain, in particular death of nerve cells, can occur. The brain pressure, i.e. the pressure prevailing in the interior of the cranium, including cerebrospinal chambers (also called intracranial pressure, ICP), is important in this context, because the blood flow through the brain and thus the oxygen and nutrient supply is influenced thereby. Under “normal” conditions, the brain pressure is permanently below approximately 10 mmHg, thus does not lead to any restriction of the blood flow through the brain, because the tissue of the brain has a natural elasticity in order to absorb blood. If the brain is damaged by an injury or a bleeding, it usually swells. However, since the brain is surrounded by a hard bone shell, which protects the brain from damage, but due to its rigid characteristics can yield only slightly during a swelling of the brain, the brain pressure can increase during a swelling, for example permanently above 15 mmHg. If the brain pressure increases, the elasticity of the brain tissue decreases, i.e. the tissue in the brain can expand less during a systolic phase and diastolic phase and less oxygen-rich blood reaches the brain. As a result, the blood flow through the brain decreases. If in the extreme case the brain pressure exceeds a limit value, i.e. is abnormal, the brain is only poorly flown through by the lack of elasticity of the tissue, and nerve cells in the brain can die.

Therefore, it is important to be able to determine whether the brain pressure lies in a normal or abnormal range, i.e. has a enough elasticity for absorbing oxygen-rich blood, in order to be able to react accordingly.

According to the currently prevailing standard, the brain pressure is determined invasively by means of pressure measuring probes in the cranial cavity (so-called gold standard of the brain pressure measurement). In this case, the pressure measuring probes are usually inserted surgically via a drainage catheter through the outer bone shell of the cranium into the cranial cavity, preferably into a lateral ventricle of the brain. By directly inserting the pressure measuring probes into the brain, the currently prevailing pressure within the cranium can be measured directly by the pressure measuring probes. The brain pressure and thus the elasticity of the brain, i.e. the property of the brain to absorb oxygen-rich blood, can be determined from the change in the measured pressure within the cranium during one or more diastolic phases and systolic phases. Furthermore, at the same time as the insertion of the brain pressure measurement, a possible liquor drainage, which is available as an effective brain pressure-lowering therapy, can be placed into the cranium. Such invasive methods are associated with considerable stresses for the patient during the operation, can lead to complications, and require appropriate follow-up care. Furthermore, such invasive methods place high demands on equipment and personnel in medical facilities.

Previous approaches to improving the gold standard of the brain pressure measurement aim above all to improve the sensitivity of the pressure measuring probes and to reduce the dimensions of such measuring probes, or to improve their contacting. In particular by reducing the size of the pressure measuring probes and additionally inserting a liquor drainage into the cranium during the surgical intervention, it was thus possible to reduce the surgical effort in the detection and treatment of an elevated brain pressure. As a result, however, the severity of the intervention in the patient could only be reduced somewhat.

Furthermore, non-invasive methods are known from WO 2020/219 773 A1 in which a transmitter and a receiver are attached to opposite sides of the skull. A distance and a propagation time of a sound signal are determined between the transmitter and the receiver. The brain pressure is estimated from the correlation of distance and propagation time. However, the brain pressure cannot be reliably determined with the aid of such methods.

Against this background, the invention is based on the object of providing a particularly simple method with which an elevated brain pressure can be determined.

This object is achieved by a computer-implemented method for classifying the brain pressure according to the main claim, wherein the method comprises: receiving data comprising time-of-flight values, which are based on time-of-flight values measured at successive time points for one or more ultrasound signals through a cross-section of a skull; classifying a characteristic of the brain pressure by means of one or more features of a representative curve of a time course of the time-of-flight values to obtain a classified characteristic of the brain pressure; and outputting the classified characteristic of the brain pressure. All method steps of the computer-implemented method described herein are carried out by one or more computers.

The time-of-flight values of the one or more ultrasound signals may have been measured by means of ultrasound probes before receiving the data, so that the presence of the patient is not required for the method steps carried out by the computer. The data can be supplied to the computer, for example, by means of a data carrier, such as a USB stick, or can be transmitted by means of a wireless or wired line. The data are processed separately without physical contact with the patient.

The invention is based on the finding of the inventors that a non-invasive assessment of the brain pressure is made possible by the method according to the invention. On the basis of the present method, the brain pressure, in particular the finding of whether it is normal or abnormal, can be determined non-invasively. This means, in particular, that no surgical intervention is necessary for the finding of whether a brain pressure is normal or abnormal, as a result of which the stress on the patient is reduced and the medical personnel and logistical effort, and ultimately the cost factor, can be significantly reduced.

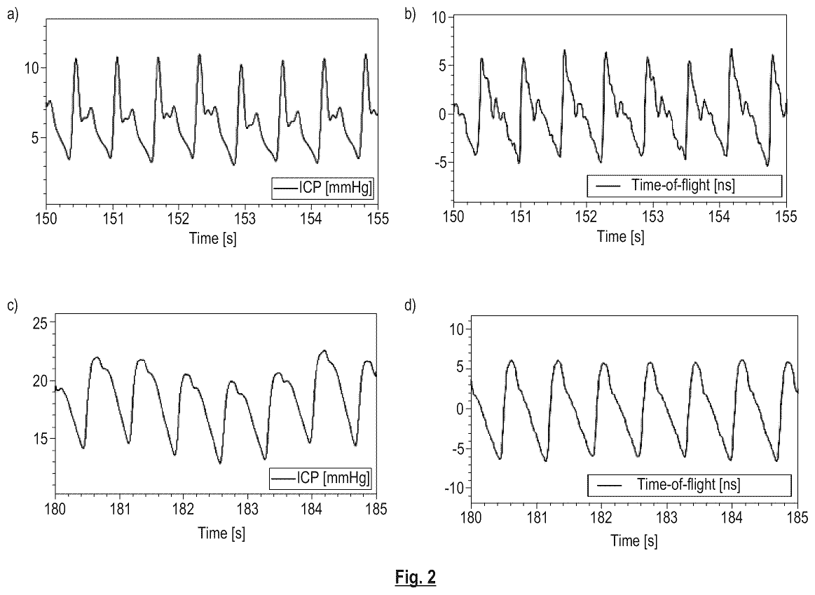

The invention is based in this connection, in particular, on the finding of the inventors that a change of a state of the brain pressure within the cranium correlates directly to a change of a time-of-flight of the one or more ultrasound signals through a cross section of the cranium (as will also be explained in more detail in connection with FIG. 2).

The heartbeat induces a pulse wave, by which blood is pressed into the vessels of the brain. The pulse wave induced by the heartbeat leads to a change in volume or expansion of the vessels in the brain, which receive the blood pressed into the brain. This change in volume leads to a change in pressure in the cranium, which has hitherto been measured directly according to the gold standard via the measuring waves in the brain.

The inventors have now recognized that a time-of-flight value of the one or more ultrasound signals measured through the cross section of the cranium is dependent on a superposition of all tissue layers and liquids located within the sound field and is thus correlated thereto.

The time-of-flight values of one or more ultrasound signals measured at successive time points through a cross section of a cranium thus detect the time-of-flight differences, which result from the pressure-induced change in volume and a pressure-induced compression of the constituents of the brain, such as blood, cerebrospinal fluid and tissue, for example.

The curve of the time course of the time-of-flight values measured at successive time points thus maps the change in volume and density of the constituents of the brain and thus of the brain pressure during one or more pulse waves.

On the basis of the features of the curve of the time course of the time-of-flight values, which reflect the pressure-induced change in volume and the pressure-induced compression of the constituents of the brain during one or more heartbeats, the brain pressure can thus be determined or classified, in particular a characteristic, for example whether it is normal or abnormal or abnormal, can be classified non-invasively.

In particular, it is made possible by the method according to the invention to determine the state within the cranium, specifically the brain pressure, without distortion by the heartbeat. A distance measurement between the probes is thus not required.

In a learning phase, the underlying algorithm for classifying a characteristic of the brain pressure was trained such that individual features of the representative curves can be assigned to a characteristic of the brain pressure.

During the learning phase, time-of-flight values were measured by the cranium in a time-dependent manner and, parallel thereto, direct brain pressure measurements were determined according to the proven gold standard on a plurality of patients with normal and abnormal brain pressure. In the selection of the patients, the following physiological inclusion criteria were taken into account: pathology: severe cranial-brain trauma (Glascow Coma Scale<10); heart rate range: 50 min-1<HF<120 min-1; pulse pressure (difference between systolic and diastolic blood pressure)<90 mmHg; systolic blood pressure<190 mmHg; diastolic blood pressure>30 mmHg.

On the basis of the features of a plurality of representative curves from time courses of the time-of-flight values in comparison with the direct brain pressure measurements, which were recorded parallel to the time-of-flight value measurements on the same patient, it was thus possible to determine whether a feature in the curves is characteristic of a particular brain pressure or whether a feature reliably separates the data from abnormal and normal brain pressures. Within the training dataset, features were identified which showed the greatest importance for the separation between elevated ICP (e.g. ≥15 mmHg) and normal ICP (e.g. ≤10 mmHg).

During the learning phase, various features on a plurality of curves were taken into account. The features taken into account can be divided into four groups: statistical features (for example mean values, standard deviation, skewness of the distribution); features from the data aggregation (for example minimum time-of-flight values, maximum time-of-flight values, average amplitudes); features from curve discussions (for example peak width, splines, extreme values, inflection points); frequency domain features from public expert library (such as tsfresh) for time-dependent signals.

During the learning phase, it was thus determined which of the examined features in the representative curves from the respective time courses are characteristic of a particular brain pressure or a normal or abnormal brain pressure, which was determined with the gold standard.

By determining these features of the curve of the time course of the time-of-flight values measured through the cranial cross section, features of a characteristic of the brain pressure can be assigned, as a result of which the brain pressure can be classified in a non-invasive manner from the curve of a time course of the time-of-flight values of one or more ultrasound signals measured through the cranial cross section.

In the time course, at least one time-of-flight value is assigned to one of the successive time points. In other words, the time course can be described as a curve in a two-dimensional coordinate system with the time-of-flight values on one axis and the respective successive time points on the complementary axis.

In one aspect, the time course of the time-of-flight values correlates to a time course of the brain pressure within the cranium.

Since the inventors have recognized that the time course of the time-of-flight values measured at successive time points correlates to the change in volume and density of the constituents of the brain and thus directly maps the time course of the pressure in the cranium, the characteristic of the brain pressure can be classified non-invasively on the basis of the curve of the time course of the time-of-flight values, in particular it can be derived whether the brain pressure is normal or abnormal or abnormal.

In one aspect, the time course of the time-of-flight values comprises a plurality of peaks, which each correlate to an increase and a decrease of the brain pressure during a systolic and a diastolic phase.

A peak is formed by an enlargement or extension of the time-of-flight values of the one or more ultrasound signals measured at successive time points through the cranial cross section. As a result, a peak correlates to an increase of the brain pressure during a heartbeat. A longer time-of-flight value is formed by an increase of the brain pressure during a systolic phase, i.e. a pressure increase in the cranium. A shorter time-of-flight value is formed by a drop of the brain pressure during a diastolic phase, i.e. a pressure drop in the cranium. A rising region of a peak thus corresponds to a systolic phase, in which blood is pressed into the vessels of the brain, and a falling region of the peak corresponds to a diastolic phase, in which blood flows out of the brain.

The time course of the time-of-flight values thus maps a plurality of systolic and diastolic phases, i.e. a plurality of successive pulse waves, in which blood is pressed into the vessels of the brain. A plurality of pulse waves are thus resolved by the time course of the time-of-flight values. As a result, the brain pressure can be reliably classified on the basis of the curve of the time course.

In one aspect, a change between time-of-flight values in the time course at at least two successive time points correlates directly to a change of the brain pressure at the at least two successive time points.

Since the inventors have recognized that a change between time-of-flight values of the one or more ultrasound signals correlates directly to a change of the brain pressure at the at least two successive time points, the brain pressure can be classified non-invasively by the method according to the invention.

In one aspect, the classified characteristic of the brain pressure indicates that a normal brain pressure, in particular a brain pressure of S1 or lower, is present or that an abnormal brain pressure, in particular a brain pressure of S2 or higher with S2≥S1, is present. The method according to the invention can distinguish between normal and abnormal brain pressures by means of different threshold values S1, S2 and is therefore not restricted to specific values for S1 and S2. The threshold values S1, S2 are determined by means of the patient population, wherein S1≤S2 applies. These threshold values S1, S2 are each usually between 5 mmHg and 20 mmHg, wherein the threshold values S1=10 mmHg and S2=15 mmHg are particularly preferred for avoiding the classification of actually abnormal brain pressures as normal.

The brain pressure can thus be classified into a normal brain pressure or an abnormal brain pressure. The corresponding finding of a normal or abnormal brain pressure can thus be output by the method; for example, the classification can be displayed on a display or can be passed on to a further entity in the form of data. On the basis of the finding, a diagnosis of a disease present at the patient can be carried out by medical personnel (without the presence of the patient).

At a brain pressure which is preferably lower than approximately 10 mmHg, an elasticity of the brain sufficient for absorbing oxygen is present. At a brain pressure which is preferably 15 mmHg or more, it is assumed that the brain pressure lies in an elevated range. In case of doubt (e.g. due to measurement inaccuracies), if, for example, the brain pressure lies between 10 and 15 mmHg, an elevated brain pressure is inferred.

In one aspect, the representative curve comprises at least one characteristic peak.

The characteristic peak is thus characteristic for the time-of-flight changes of a plurality of pulse waves in the brain. For example, the characteristic peak can be formed by forming a median of the time-of-flight values of a plurality of peaks in the time course of the time-of-flight values. In other words, the characteristic peak in the representative curve maps a characteristic increase and a characteristic decrease of the brain pressure of a plurality of systolic and a plurality of diastolic phases. Thereby, the at least one characteristic peak better maps a characteristic behavior of the brain. The brain pressure can thus be more reliably classified on the basis of the time course of the time-of-flight values.

In one aspect, the characteristic of the brain pressure is determined by the computer by extracting the one or more features of the curve of the at least one characteristic peak.

The characteristic peak reflects the time-of-flight changes over a plurality of pulse waves in the brain and thus maps the behavior of the brain more meaningfully. By extracting the one or more features from the curve of the at least one characteristic peak, a more reliable classification is thus ensured.

In one aspect, a first feature of the one or more features is a peak width of the at least one characteristic peak at a certain percentage of the maximum amplitude of the at least one characteristic peak.

In the course of the learning phase, the inventors have found that a feature which can be assigned to a normal or abnormal brain pressure is the peak width in the time course of the time-of-flight values. Thus, a normal or abnormal brain pressure can be determined on the basis of the peak width of the at least one characteristic peak at a certain percentage of the maximum amplitude.

In one aspect, a second characteristic feature of the plurality of features is a similarity of the curve shape of the at least one characteristic peak to a synthetic function, in particular a continuous wavelet transform.

In addition, in the course of the learning phase, the inventors have found that a further feature which reflects a normal or abnormal brain pressure is a similarity of the curve shape of the at least one characteristic peak to a synthetic function.

In the course of the learning phase, a plurality of characteristic peaks were convoluted or overlaid with a plurality of defined synthetic functions, wherein various synthetic functions were tried out. The similarity of the curve shape can be determined on the basis of the mathematical convolution of the curve shape of a synthetic function. Various wavelet functions were used as synthetic functions, wherein properties of the wavelet functions were varied by scaling factors, such as, for example, their coefficients, and the corresponding functions were compressed and expanded in this way. Examples of wavelet functions used are: the db4, db16, haar, coif, sym4, sym8, bior1.3 or the bior3.1 functions. Descriptions for wavelet functions can be retrieved, for example, at: https://de.mathworks.com/help/wavelet/ref/cwt.html or at https://en.wikipedia.org/wiki/Ricker_wavelet.

In this case, by means of a comparison with corresponding brain pressure measurements according to the gold standard, the inventors have found that certain scaling coefficients of the wavelet functions reflect a normal or abnormal brain pressure.

By means of the second characteristic feature, which is extracted from the representative peak, the characteristic of the brain pressure can be reliably classified. A particular reliability of the classification can be achieved by a combination of the first and second features being determined from the at least one characteristic peak.

In one aspect, the at least one characteristic peak is for a plurality of peaks in at least one time segment of the time course of the time-of-flight values.

According to this aspect, the at least one characteristic peak is formed on the basis of a time segment, i.e. a time segment, of the time course of the time-of-flight values. In other words, a segment can be understood as a time segment which contains the time-of-flight values which are assigned to a subgroup of time points of the successive time points.

For example, a segment with the best quality of the data of the time-of-flight values can be selected in this way. In other words, segments with poor quality of the data of the time-of-flight values can be excluded in this way. As a result, the classification of the characteristic of the brain pressure becomes more reliable. In addition, the computation capacity required can be reduced by the associated reduction in the data used for the evaluation.

In one aspect, the at least one characteristic peak is determined by the following steps: extracting a plurality of individual peaks from the at least one time segment of the time course of the time-of-flight values; normalizing time points of the time-of-flight values in the individual peaks; grouping the individual peaks by applying a similarity criterion for the individual peaks; identifying the group with the largest number of peaks, and forming the at least one characteristic peak from the peaks in the identified group of peaks.

The at least one characteristic peak is thus formed from the time-of-flight values of the peaks from a group of the most peaks which meet the similarity criterion.

The similarity criterion can comprise, for example, forming a silhouette coefficient with respect to the individual peaks.

Thereby, the at least one characteristic peak maps a plurality of peaks with relatively meaningful data quality. As a result, the classification of the characteristic of the brain pressure by means of the measured time-of-flight values becomes more reliable.

In one aspect, forming the at least one characteristic peak comprises: for each time point, forming a median of the time-of-flight values of the peaks from the identified group.

Thereby, the time-of-flight values of the at least one characteristic peak are formed by the median of the time-of-flight values of a plurality of peaks. Thereby, the at least one characteristic peak maps a plurality of peaks. As a result, the classification of the characteristic of the brain pressure by means of the measured time-of-flight values becomes more reliable.

In one aspect, the at least one segment is determined by the following steps: extracting one or more time segments from the time course of the time-of-flight values, wherein a segment comprises a plurality of peaks in the time-of-flight values; determining the at least one time segment by selecting one or more time segments in which the peaks meet a quality criterion.

According to this aspect, such segments are selected in which the curve of the time-of-flight values meet a certain quality. Thereby, time segments in the measured time-of-flight values, which contain, for example, disturbances due to heart failures or the like, can be excluded and are not used for further data processing. By selecting individual segments from the time course of the time-of-flight values for classification according to a quality criterion, the classification of the characteristic of the brain pressure becomes more reliable. Furthermore, the computation effort in the evaluation of the data can thus be reduced.

In one aspect, the quality criterion is formed by an autocorrelation function which evaluates a self-similarity of the time course of the time-of-flight values in a segment.

The autocorrelation function is a measure for the self-similarity of the curve. Since the time course of the time-of-flight values is characterized by rhythmic, recurring fluctuations with high similarity, the autocorrelation function represents a criterion for the presence of a brain pulse curve.

According to this aspect, segments in which the peaks in the curve of the time course of the time-of-flight values comprise disturbances, which correspond, for example, to a non-present heartbeat, can be excluded. As a result, the reliability of the classification of the characteristic of the brain pressure is improved. In one aspect, the quality criterion is formed by a comparison of the maximum amplitudes of the most dominant frequency component with amplitudes of further frequency components in the time course of the time-of-flight values in a segment.

In other words, in this aspect, the quality criterion can be described as a Peak2mean criterion which describes the ratio between the amplitude of the pulse and the harmonics thereof to the remaining frequency components in the time course of the time-of-flight values. This is thus an operation in the frequency domain. Peak2mean is a measure for the fact that the brain pulse curve represents the dominant component in the time course and is afflicted with a correspondingly high amplitude. If the signal-to-noise ratio is too low, the respective segment is not used for a further analysis.

According to this aspect, segments in which the peaks in the curve of the time course of the time-of-flight values comprise disturbances, by which the peaks caused by the heartbeat appear as the non-most dominant component, can be excluded. As a result, the reliability of the classification of the characteristic of the brain pressure is improved.

In one aspect, the quality criterion is formed by an evaluation of frequencies in the time course of the time-of-flight values in a segment.

The quality criterion of this aspect can be designated from the upper band noise criterion which describes the component of higher frequencies (≥15 Hz) which are not generated by harmonics of the heartbeat. A high component is a sign of increased noise; the respective segment is not used for a further analysis. Noise amplitudes in this frequency range are not to be assigned of physiological origin.

By this aspect, segments in which the peaks in the curve of the time course of the time-of-flight values comprise disturbances, which are caused, for example, by non-physiological noise, can be excluded. Furthermore, background frequencies which are not caused by the heartbeat can be excluded.

The quality criteria of these aspects can be applied individually or also in combination for selecting a segment. By a combination of a plurality of quality criteria, segments with particularly meaningful data quality can be selected.

Thus, for example, a segment can be selected which meets the quality criterion formed by the autocorrelation function and additionally meets the quality criterion formed by the comparison of the maximum amplitudes of the most dominant frequency component or meets the quality criterion formed by the evaluation of frequencies in the time course.

In other aspects, for example, a segment can be selected which meets the quality criterion formed by the evaluation of frequencies in the time course and additionally meets the quality criterion formed by the comparison of the maximum amplitudes of the most dominant frequency component.

In particularly preferred aspects, a segment can be selected which meets the quality criterion formed by the autocorrelation function and meets the quality criterion formed by the comparison of the maximum amplitudes of the most dominant frequency component and meets the quality criterion formed by the evaluation of frequencies in the time course. This aspect is particularly preferred since in this way a segment can be selected which meets the quality criteria of all three aspects and thus provides a particularly meaningful data quality of the one or more selected segments.

In one aspect, the method further comprises: measuring the time-of-flight values of the one or more ultrasound signals through the cross-section of the skull by means of at least two probes positioned on opposite sides of the skull at the successive time points.

According to this aspect, the propagation time of the one or more ultrasound signals can be measured. A time-of-flight value corresponds to the time-of-flight required by the one or more ultrasound signals to traverse the skull between the probes along the cross-section.

To measure the time-of-flight, the at least two probes are positioned on opposite sides of the skull. The at least two probes can comprise at least one emitter which is configured to output the one or more ultrasound signals, and at least one detector which is configured to receive the one or more ultrasound signals output by the at least one emitter. The at least one emitter and the at least one detector can be positioned on different opposite sides of the skull such that a sound field between the probes is approximately parallel to a frontal cross-section of the skull. By means of the at least one emitter and the at least one detector, the time-of-flight required by the one or more ultrasound signals to pass from the at least one emitter to the at least one detector through the cross-section of the skull can thus be measured.

As was described at the outset, by measuring the time-of-flight of the one or more ultrasound signals, a characteristic of the brain pressure can be determined and classified non-invasively, since the time-of-flight of the one or more ultrasound signals images a state of the brain along the cross-section in the skull.

Furthermore, the one or more ultrasound signals can be a longitudinal wave. The inventors have found that this type of wave is particularly well suited to determining and classifying the brain pressure.

The probes comprise at least one emitter for outputting the one or more ultrasound signals and at least one corresponding detector for detecting the output one or more ultrasound signals, wherein the at least one emitter and the at least one detector are positioned on the opposite sides of the skull.

By means of this positioning, the time-of-flight through the cross-section along the skull can be measured.

The emitter and the detector are each positioned in a region above the outer ear canal.

The inventors have found that the positioning of the emitter and of the detector on opposite regions of the skull above the ear canal is particularly advantageously suited to imaging the internal state of the brain and, as a result, the characteristic of the brain can be classified particularly reliably.

The time-of-flight values of the one or more ultrasound signals through the skull can be measured by: outputting a plurality of wave packets each having an n-periodic signal of constant frequency at respective one of the successive time points; measuring the time-of-flight values through the cross-section of the skull for each of the n-periods of the periodic signal at each of the plurality of wave packets.

As a result, a particularly advantageous measuring method is disclosed in order to provide a sufficient number of time-of-flight values for processing, wherein the invention is not restricted to this specific measuring method. A wave packet is output at a time point by a probe, in particular the emitter. A wave packet comprises a periodic signal having n-periods, wherein n is an integer. The wave packet passes through the cranial cross section and is detected at the probe on the opposite side of the skull. A time-of-flight value is determined at each of the n-periods of the wave packet. This means that n time-of-flight values can be assigned to each time point.

As a result, the number of available measured values, which are available for later processing of the measured values, is increased. By increasing the number of measured values, the signal better describes the time course of the internal state of the brain pressure in the brain. As a result, it is possible to realize a more reliable evaluation of the measured time-of-flight values. Ultimately, the classification of the characteristic of the brain pressure is thus more reliable.

In one aspect, receiving data with time-of-flight values comprises: receiving a number of n time-of-flight curves, wherein in each of the n time-of-flight curves a time-of-flight value is assigned to one of the successive time points; for each of the n time-of-flight curves, subtracting an offset from the time-of-flight values in the time-of-flight curve;

forming an averaged time-of-flight curve by averaging the time-of-flight values of each of the n time-of-flight curves at one of the successive time points.

In a further aspect, in one of the n time-of-flight curves a time-of-flight value at a time point is formed by a period of an n-periodic wave packet of the one or more ultrasound signals which was output or received at the corresponding time point.

From the preceding measurement, n time-of-flight values were determined at each time point (wherein n corresponds to the number of periods in a wave packet). As a result, a number of n-temporal profiles of time-of-flight values can be formed, wherein in each of the n-temporal profiles the time-of-flight value of an identical period in a wave packet is assigned to a time point of the wave packet. Each of the temporal profiles thus forms a time-of-flight curve. Thereby, n time-of-flight curves are formed. An offset is subtracted from each of the n time-of-flight curves. The offset can map a plurality of factors which are not correlated directly to the cardiac activity. For example, frequency-dependent interference signals from the measuring apparatus or the respiration of a patient can be subtracted by the offset. An averaged time-of-flight curve is formed by averaging the time-of-flight values at corresponding time points of the n time-of-flight curves.

This averaged time-of-flight curve can be used as a basis for further processing of the data, for example, by forming the one or more segments and/or forming the averaged peak.

By means of this aspect, the measured data are conditioned in such a way that the data are freed of a background not correlated directly to the cardiac activity and, by means of the averaging, reproduce a meaningful map of the changing state of the brain along the cross-section. As a result, a morphology in the time course of the measured time-of-flight value can be better worked out. The classification by means of the time course of the time-of-flight values thus becomes more reliable.

In one aspect, each of the n time-of-flight curves has a periodicity which is given by the heart rate.

The measured values received at the computer thus reflect the change in the brain pressure induced by the heartbeat.

In one aspect, the one or more time segments are selected from the averaged time-of-flight curve for determining the at least one segment.

The averaged time-of-flight curve is thus used as a basis for the time segments for determining the at least one characteristic peak. As a result, a better and more reliable data quality for determining the at least one characteristic peak is achieved, as a result of which the characterization of the characteristic of the brain pressure becomes more reliable.

In one aspect, a bandpass filter is applied to the time-of-flight values in a time-of-flight curve for determining the offset.

Low-frequency frequency components (for example a trend or breathing effects) can be cut off by the bandpass filter. As a result, frequency components not correlated directly to the cardiac activity can be cut off, as a result of which the classification of the characteristic of the brain pressure can be realized more reliably.

Furthermore, the object mentioned at the outset is achieved by an apparatus, in particular a computer, configured to carry out the method according to the invention for classifying a brain pressure. The computer can comprise one or more processors which are configured to carry out the method according to the invention. Furthermore, the computer can comprise a storage medium on which instructions are stored which, when they are executed by the one or more processors, cause the one or more processors to carry out the method according to the invention.

Furthermore, the object mentioned at the outset is achieved by an apparatus arrangement comprising the apparatus described above and at least two probes for determining the time-of-flight values of one or more ultrasound signals through a cross-section of a skull, wherein the at least two probes comprise at least one emitter for outputting the one or more ultrasound signals and at least one corresponding detector for detecting the output one or more ultrasound signals, wherein in particular the at least one emitter and the at least one detector can be positioned on opposite sides of the skull, in particular in a plane through a frontal cross-section of the skull.

In one aspect, the at least one emitter and the at least one detector can each be positioned in a region above the outer ear canal.

In one aspect, the at least two probes are configured to output a plurality of wave packets each having an n-periodic signal of constant frequency at respective one of the successive time points, and to detect time-of-flight values through the cross-section of the skull for each of the n-periods of the periodic signal at each of the plurality of wave packets.

Furthermore, the object mentioned at the outset is achieved by a computer program, comprising instructions which, when the method according to the invention is executed by a computer, cause the computer to carry out the method according to the invention and a computer-readable medium on which the computer program is stored.

Further properties, features and advantages of the invention will become clear below by means of a description of preferred embodiments of the invention with reference to the accompanying exemplary drawings, in which:

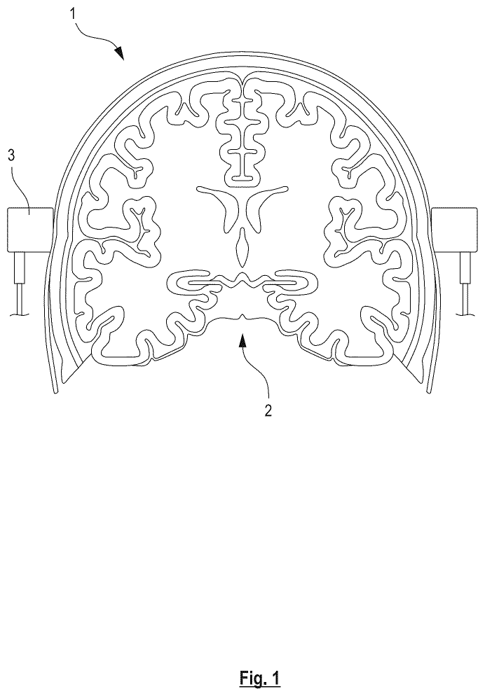

FIG. 1 shows by way of example a measurement arrangement during the determination of the time-of-flight values of one or more ultrasound signals through a skull.

FIG. 2 shows a verification of the measured temporal profiles of the time-of-flight values.

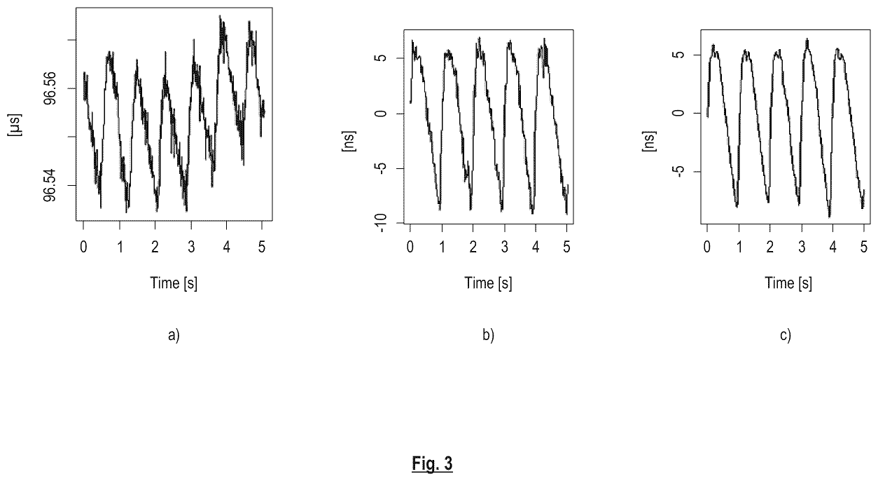

FIG. 3 shows by way of example the directly measured values and the preprocessing thereof.

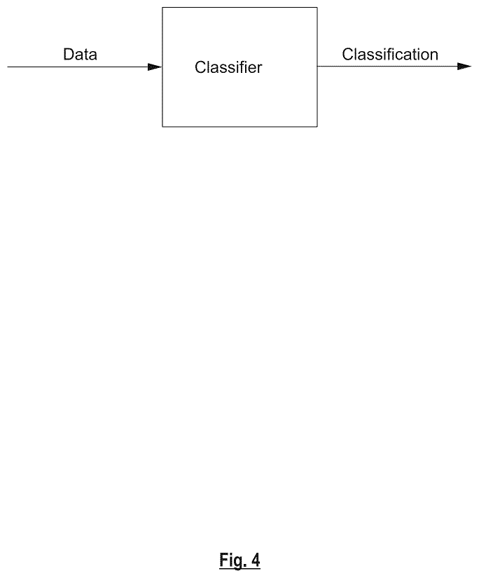

FIG. 4 shows by way of example a classifier.

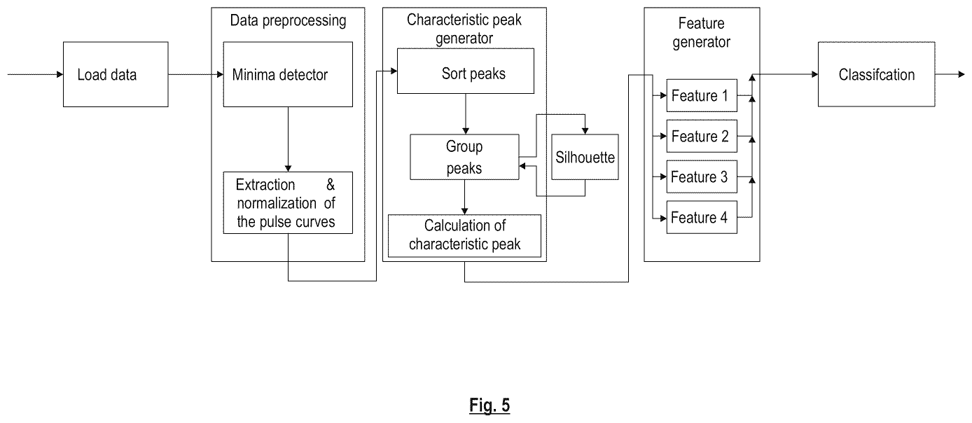

FIG. 5 shows by way of example a detailed view of the classifier.

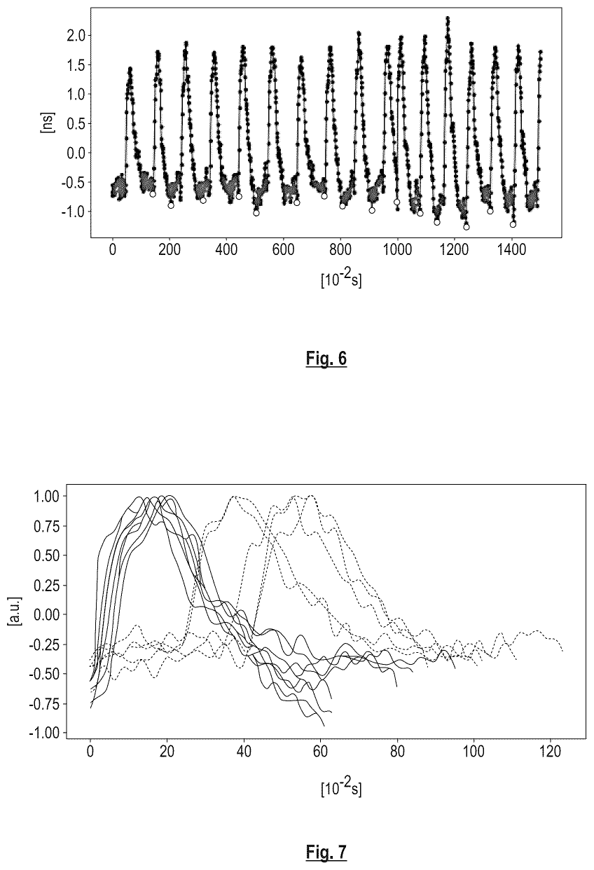

FIG. 6 shows by way of example the determination of minima on a data set.

FIG. 7 shows by way of example a grouping of individual peaks on a data set.

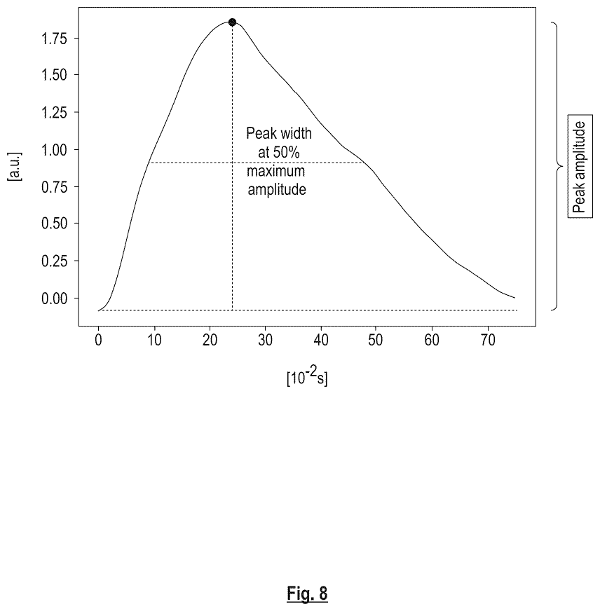

FIG. 8 shows by way of example a characteristic peak.

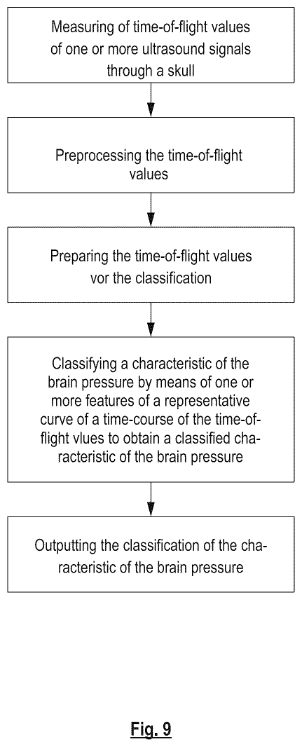

FIG. 9 shows by way of example a temporal sequence of the method steps.

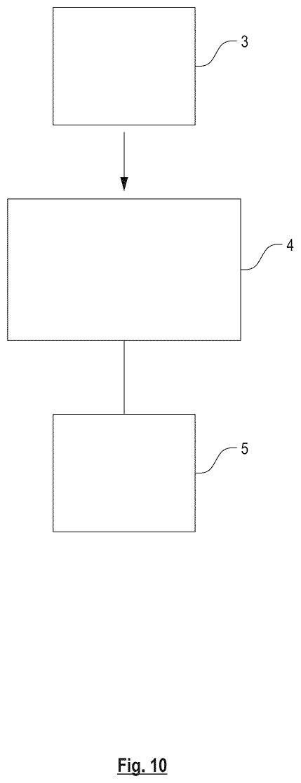

FIG. 10 shows by way of example an arrangement for classifying the brain pressure.

The features disclosed in the above description, the figures and the claims can be important both individually and in any combination for the realization of the invention in the various configurations.

Reference signs in the figures relate in each case to same elements.

FIG. 1 shows by way of example a measurement arrangement for determining the time-of-flight values of one or more ultrasound signals through a cranial cross section.

Two probes are shown, which are positioned on opposite sides of the skull 1 with a brain 2. The two probes 3 comprise at least one emitter which is configured to output the one or more ultrasound signals, and at least one detector which is configured to receive the one or more ultrasound signals output by the at least one emitter. The one or more ultrasound signals can be implemented by a longitudinal wave. Furthermore, the one or more ultrasound signals can have one or more wave packets with constant frequency. The at least one emitter and the at least one detector are positioned on the different opposite sides of the skull 1.

Between the emission and the detection, the one or more ultrasound signals traverse the cross-section of the skull 1. A sound field which covers a certain region of the cranial cross section and which traverses the one or more ultrasound signals is thus formed between the emitter and the detector. The sound field can preferably be aligned parallel to a frontal plane of the skull 1.

As was described at the outset, the propagation time of the one or more ultrasound signals measured at different time points depends on a change in the internal state of the brain pressure of the brain 2. A change in the time-of-flight values between two successive time points thus maps a change in the state of the components in the skull 1.

A verification of the measured time-of-flight values by means of a comparison of the measured time-of-flight values with a time course, measured simultaneously on the same patients, of the brain pressure measured according to the gold standard is explained below.

FIG. 2 shows, in sections (a) and (c), brain pressure measurements which were carried out by means of the measurement method according to the gold standard of the brain pressure measurement on a patient with normal brain pressure (section (a)) and abnormal brain pressure (section (c)). Furthermore, FIG. 2 shows, in sections (b) and (d), time-of-flight measurements which were carried out simultaneously with the brain pressure measurements on the same patients with normal brain pressure (section (b)) and abnormal brain pressure (section (d)). The time-of-flight values shown have in this case been measured and preprocessed according to the method described in more detail in FIG. 3.

The brain pressure measurements shown in sections (a) and (c) and carried out according to the gold standard serve as a reference. As was described at the outset, in the gold standard of the brain pressure measurements, pressure measuring probes are inserted into the brain 2 and the brain pressure is thus measured directly, i.e. relatively reliably. This measuring method is generally recognized and can thus serve as a reference.

In section (a) of FIG. 2, a time course of the directly measured brain pressure (ICP) on a patient with normal brain pressure is illustrated. The time course of the brain pressure comprises a characteristic curve which comprises a plurality of peaks, which each map a pressure increase during a systolic phase and a pressure decrease during a diastolic phase. These peaks evidently have a continuously specific shape, which can be assigned to an elasticity of the brain 2 of the patient in a normal range, as a result of which a brain pressure in a normal range can be reliably inferred.

In section (c) of FIG. 2, a time course of the directly measured brain pressure (ICP) on a patient with abnormal brain pressure is illustrated. The time course of the brain pressure likewise comprises a characteristic curve which comprises a plurality of peaks correlating to a systolic and diastolic phase. As can be gathered from section (c) of FIG. 2, the curve has a significantly changed shape compared with the curve of the time course of the brain pressure in a patient with normal brain pressure. An abnormal brain pressure can thus be reliably inferred from the curve shape.

A normal or abnormal brain pressure can thus be reliably inferred from the curves of the time courses of the brain pressure measurements according to the gold standard.

In contrast, in section (b) of FIG. 2, a time course of the time-of-flight values, measured by means of the described arrangement, of one or more ultrasound signals through the cranial cross section which were determined simultaneously on the same patient with normal brain pressure as in section (a) is shown. The time course of the time-of-flight values likewise comprises a characteristic curve with a plurality of peaks, which can thus likewise be assigned to a systolic and diastolic phase. As can be seen, the curve of the time-of-flight values has a very similar shape to the curve of the time course of the brain pressure shown in section (a). It can thus be reliably concluded that the curve of the time course of the time-of-flight values correlates directly to the change of the brain pressure in the skull 1 and a normal brain pressure of the patient can be inferred by means of the curve of the time course of the time-of-flight values.

In section (d), a time course of the time-of-flight values, measured by means of the described arrangement, of one or more ultrasound signals through the cranial cross section which were determined simultaneously on the same patient with abnormal brain pressure as in section (c) is shown. The time course of the time-of-flight values likewise comprises a characteristic curve with a plurality of peaks, which can thus likewise be assigned in each case to a systolic and diastolic phase. As can be seen, the curve of the time-of-flight values has a very similar shape to the curve of the time course of the brain pressure shown in section (c). It can thus be reliably concluded that the curve of the time course of the time-of-flight values correlates directly to the change of the brain pressure in the skull 1. An abnormal brain pressure of the patient can thus be reliably inferred by means of the curve of the time course shown in section (d).

These simultaneous measurements were repeated on a plurality of patients with normal and abnormal brain pressures and, in principle, similar correlations of the time courses of the brain pressure measured according to the gold standard to the time courses of the time-of-flight values were observed. In the selection of the patients, the physiological inclusion criteria mentioned at the outset were used as a basis.

A normal or abnormal brain pressure can thus be reliably inferred by means of the curve of the time course of the time-of-flight values measured at successive time points.

This finding can be explained by a change in a state of the brain pressure within the skull 1 correlating directly to a change in the speed of sound and thus to a time-of-flight of the one or more ultrasound signals through the cross section of the skull 1.

The time-of-flight values of one or more ultrasound signals measured at successive time points through a cross section of a skull 1 thus detect the time-of-flight differences, which result from the pressure-induced change in volume and a pressure-induced compression of the constituents of the brain 2, such as blood, cerebrospinal fluid and tissue, for example.

The time course of the time-of-flight values measured at successive time points thus reflects the change in volume and density of the constituents of the brain 2 and thus of the brain pressure during a pulse wave, as a result of which a brain pressure can be classified reliably non-invasively.

A correlation of the time-of-flight values of one or more ultrasound signals through the brain to an internal state of the brain pressure of the brain 2 is scientifically proven.

In connection with FIG. 3, the measurement of the time-of-flight values of the one or more ultrasound signals through the brain 2 and the subsequent preprocessing of the data of the measured time-of-flight values are explained in more detail.

It should be noted that the measuring method and the preprocessing of the measured measured values are shown merely by way of example, and other measuring methods and data preprocessings are also possible in order to make possible a sufficient data quality for the classification of the brain pressure by means of a time course of time-of-flight values. Thus, for example, other measuring methods can be applied which provide a sufficient data quality, in which corresponding data processing is not necessary for determining an evaluable time course.

A wave packet of constant frequency of the one or more ultrasound signals is output at one of the successive time points by the emitter. A wave packet comprises a periodic signal having n-periods, wherein n is an integer. The wave packet passes through the cross-section of the skull 1 and is detected at the detector on the opposite side of the skull 1. At each of the n-periods of the wave packet, a time-of-flight value of a time-of-flight from the emitter to the detector is determined. This means that n time-of-flight values are assigned to each time point.

From these measurements, n time-of-flight values are determined at each time point, wherein a time point is assigned to each of the n time-of-flight values. Then, a number of n temporal profiles of time-of-flight values is formed, wherein in each of the n temporal profiles a time-of-flight value of an identical period in a wave packet is assigned to a time point of the wave packet. Each of the n temporal profiles thus forms a time-of-flight curve. Thus, n time-of-flight curves are formed according to the number of n-periods in a wave packet.

FIG. 3(a) shows one of the n time-of-flight curves with the directly measured time-of-flight values, wherein the time points are specified in units of seconds on the X-axis and the time-of-flight values in microseconds on the Y-axis. In the time-of-flight curve, a time-of-flight value of a period of the periodic signal of a wave packet is assigned to each of the successive time points. The time-of-flight values fluctuate over the shown approximately 5 seconds due to the heartbeat. Furthermore, n−1 further time-of-flight curves (not shown) exist, wherein in each of the n−1 time-of-flight curves a time point assigned by the same period is assigned to the corresponding time point.

An offset is subtracted from each of the n time-of-flight curves. The offset is subtracted by applying a bandpass filter to the time-of-flight values in a time-of-flight curve. Low-frequency frequency components (for example a trend or breathing effects) can be cut off by the bandpass filter.

FIG. 3(b) shows by way of example the time-of-flight curve from FIG. 3(a) after subtracting an offset (e.g. constant and trend), wherein the time points are specified in units of seconds on the X-axis and the time-of-flight values in nanoseconds on the Y-axis.

An averaged time-of-flight curve is then formed by averaging the time-of-flight values of each of the n time-of-flight curves at one of the successive time points. In the averaged time-of-flight curve, an averaged time-of-flight value from the corresponding n time-of-flight values is assigned to a time point. The corresponding averaged time-of-flight curve is shown by way of example in FIG. 30, wherein the time points are specified in units of seconds on the X-axis and the time-of-flight values in nanoseconds on the Y-axis.

The averaged time-of-flight curve can be used as a basis for further processing of the data. In the case of sufficient signal quality, however, time-of-flight values which have not undergone such a measuring and processing process can also be used as a basis for further processing.

Furthermore, the temporal profiles of the time-of-flight values can run through an optional preparation step for classification. In the preprocessing step for classification, the directly measured temporal profiles of the time-of-flight values can be formed into temporal segments of successive time points with the correspondingly assigned time-of-flight values, wherein these segments are used as a basis for the data preprocessing and a plurality of segments with a correspondingly averaged time-of-flight curve are formed. In other aspects, temporal segments of successive time points with the correspondingly assigned time-of-flight values can be formed from the averaged curve and used as a basis for further processing.

One or more segments which meet a quality criterion can be selected from the segments formed in this way.

The quality criterion can be formed by an autocorrelation function which evaluates a self-similarity of the time course of the time-of-flight values in a segment. In addition or alone, the quality criterion can be formed by a comparison of the maximum amplitudes of the most dominant frequency component with amplitudes of further frequency components in the time course of the time-of-flight values in a segment. In addition or alone, the quality criterion can be formed by an evaluation of frequencies in the time course of the time-of-flight values in a segment.

The selected segments can be used as a basis for further processing, in particular can be loaded into a classifier.

FIG. 4 shows by way of example the mode of operation of a classifier for classifying the characteristic of the time course of the time-of-flight curve. The classifier is implemented by the computer.

Data comprising time-of-flight values, measured at successive time points, of the one or more ultrasound signals through a cross section of a skull 1 are loaded into the classifier. The loaded data can be data directly from successively measured time-of-flight values which have not undergone a preprocessing step, data which have undergone a preprocessing step, in particular the averaged time-of-flight curve, or the segments selected according to the quality criterion.

In the classifier, a characteristic of the brain pressure is classified by means of the one or more features of a representative curve of the time course of the time-of-flight values to subsequently output a classified characteristic of the brain pressure.

FIG. 5 shows a detailed view of the classifier to clarify the classification of the characteristic of the brain pressure.

As described, data comprising time-of-flight values measured at successive time points are loaded into the classifier.

In one aspect, a plurality of the selected segments are loaded into the classifier and combined to form a time course of the time-of-flight values. In a further aspect, the unprocessed time-of-flight values are loaded into the classifier. In an additional aspect, the averaged time-of-flight curve is loaded into the classifier.

In a corresponding time course of the time-of-flight values, individual peaks are initially determined by detecting minima in the time course between adjacent peaks. A corresponding data set is shown in FIG. 6, wherein successive time points are specified in 10\hat{ }(−2) seconds on the X-axis and the corresponding time-of-flight values in nanoseconds on the Y-axis. The detected minima are indicated by the points without filling.

In a next step in FIG. 5, the peaks determined in this way are extracted and normalized in time to a common starting time point.

In an optional subsequent step, peaks which are too short or too long can be sorted out. The sorting out of too short or too long peaks results from a determination of whether a peak width on the detected minima lies below or above a reference value which is determined by a median or mean of the peak widths of the cut-out peaks on their minima.

In one step, the cut-out and normalized peaks are grouped. The grouping is carried out by applying a similarity criterion for the individual peaks. The group of peaks which comprises the most peaks is selected for further processing.

The grouping of the cut-out and normalized peaks is shown by way of example in FIG. 7, wherein the time points normalized in time in units of 10\hat{ }(−2) seconds are shown without units on the X-axis and the time-of-flight values normalized to a maximum peak amplitude are shown without units on the Y-axis. In this example, the group of peaks with amplitude maxima in a range of approximately 2 seconds is selected for further processing (shown by means of solid lines in the curves).

By forming the median of the respective time-of-flight values of the peaks of the selected curve at a respective time point, the at least one characteristic peak of the representative curve can be formed. The median is thus formed from time-of-flight values of individual peaks at a time point. The at least one characteristic peak thus characterizes peaks of a plurality of peaks in the time course of the time-of-flight.

A peak determined in this way is shown by way of example in FIG. 8, wherein the normalized time points are specified in units of 10\hat{ }(−2) seconds on the X-axis and the normalized time-of-flight values of the characteristic peak are specified without units on the Y-axis.

Features of the curve of the at least one characteristic peak are then determined from the at least one characteristic peak.

As described at the outset, these features were determined during a learning phase. Feature of the one or more features can be, for example, a similarity of the curve shape of the at least one characteristic peak to a synthetic function, in particular a continuous wavelet transform. Furthermore, a further feature can be a peak width of the at least one characteristic peak at a certain percentage of the maximum amplitude of the at least one characteristic peak.

On the basis of one or more features of the characteristic peak, the characteristic of the brain pressure can be classified, in particular whether the brain pressure is normal or abnormal. Based on the classification, a classification can be determined and output by the computer. A diagnosis of a disease can be carried out on the basis of the output classification.

FIG. 9 shows a temporal sequence of the steps of the method described above.

In a first step, time-of-flight values of one or more ultrasound signals through a skull 1 are measured.

In a second step, the time-of-flight values thus measured can be preprocessed for further processing.

In a third step, the time-of-flight values can be prepared for the classification.

The preprocessing of the time-of-flight values and the preparation of the time-of-flight values for the classification in the second and third step are optional. For example, the measured time-of-flight values can be loaded directly into the classifier.

In a fourth step, a characteristic of the brain pressure is classified by means of one or more features of a representative curve of a time course of the time-of-flight values to obtain a classified characteristic of the brain pressure.

In a fifth step, the classification of the characteristic of the brain pressure is output.

FIG. 10 shows by way of example an arrangement for classifying a brain pressure.

The apparatus arrangement comprises two probes 3. The two probes 3 comprise an emitter for outputting one or more ultrasound signals and a detector for detecting the one or more ultrasound signals. The probes 3 can be, for example, part of a measuring apparatus. By means of the probes 3, the time-of-flight values of the one or more ultrasound signals from the emitter to the detector through the skull 1 are measured.

The measured time-of-flight values for the ultrasound signals are transmitted to a computer 4. The transmission is carried out by means of a suitable medium. For example, data with the time-of-flight values can be transmitted to the computer 4 by means of a data carrier, such as a USB stick. In other examples, the data with the time-of-flight values can be transmitted to the computer 4 in a wired manner. In further examples, the data can be transmitted to the computer 4 wirelessly, for example by means of a WLAN or Bluetooth connection. For this purpose, the probes 3 or the measuring apparatus are in a corresponding communication connection with the computer 4. The computer 4 carries out the method described above for classifying a brain pressure on the basis of the received data with time-of-flight values. The output can be carried out on a display connected to the computer 4. The classified characteristic of the brain pressure can then be displayed to a user by the display.

Furthermore, a computer-readable medium 5, such as a storage medium or a data carrier, is provided which can be connected to the computer. The computer-readable medium 5 comprises instructions which, when executed by the computer, cause the latter to carry out the steps according to the method described.

The above-described aspects and embodiments of the technology described herein can be implemented in numerous ways. For example, the embodiments can be implemented using hardware, software or a combination thereof.

If a feature is implemented in software, the software code can be executed on any suitable computer or processor or collection of processors, irrespective of whether it is provided in a single computer or distributed among a plurality of computers. Such processors can be implemented as integrated circuits, with one or more processors in an integrated circuit component, including commercially available integrated circuit components known in the art by names such as CPU chips, GPU chips, microprocessor, microcontroller or coprocessor. Alternatively, a processor can be implemented as an ASIC or as a configuration of a programmable logic device. As yet another alternative, a processor can be part of a larger circuit or semiconductor device. As a specific example, some commercially available microprocessors have a plurality of cores, such that one or a subset of these cores can form a processor. However, a processor can be implemented using circuitry in any suitable format.

Furthermore, a computer can be embodied in any of a number of forms, such as, for example, a rack-mounted computer, a desktop computer, a laptop computer or a tablet computer. Furthermore, a computer can be embedded in a device which is generally not considered to be a computer but has suitable processing capabilities, including a personal digital assistant (PDA), a smartphone or another suitable portable or permanently installed electronic device.

Such computers can be interconnected by one or more networks in any suitable form, including as a local area network or a wide area network, such as an enterprise network or the Internet. Such networks can be based on any suitable technology and can operate according to any suitable protocol, and can comprise wireless networks, wired networks or fiber-optic networks.

Furthermore, the various methods or processes outlined herein can be encoded as software which is executable on one or more processors using any of a plurality of operating systems or platforms. Furthermore, such software can be written using a number of suitable programming languages and/or programming or scripting tools and can also be compiled as executable machine language code or intermediate code which is executed on a framework or virtual machine. The terms “(computer) program” or “software” are used herein in a general sense to refer to any type of computer code or set of computer-executable instructions which can be used to program a computer or other processor to implement various aspects of the present invention as discussed above. Furthermore, it should be noted that, according to one aspect of this embodiment, one or more computer programs which, when executed, carry out methods of the present invention need not reside on a single computer or processor, but may be distributed in a modular manner among a number of different computers or processors to implement various aspects of the present invention.

The invention may be embodied as a computer-readable (memory) medium (or multiple computer-readable media) (e.g., a computer memory, one or more floppy disks, compact discs (CD), optical disks, digital video disks) (DVD), magnetic tapes, flash memory, circuit configurations in field programmable gate arrays or other semiconductor devices or other tangible computer storage media) encoded with one or more programs which, when executed on one or more computers or processors, carry out methods which implement the various embodiments of the invention discussed above. As can be seen from the above examples, a computer-readable storage medium may store information for a sufficient time to provide computer-executable instructions in a non-transitory form. Such a computer-readable storage medium or media may be portable so that the program or programs stored thereon can be loaded onto one or more different computers or other processors to implement various aspects of the present invention as discussed above. As used herein, the term “computer-readable (memory) medium” includes only a non-transitory computer-readable medium which may be considered as a physical article of manufacture (i.e., an article of manufacture) or a machine. Alternatively or additionally, the invention may be embodied as a computer-readable medium other than a computer-readable storage medium, such as a propagating signal.

Computer-executable instructions or commands may be in many forms, such as program modules, executed by one or more computers or other devices. In general, program modules comprise routines, programs, objects, components, data structures, etc., which perform particular tasks or implement particular abstract data types. Typically, the functionality of the program modules may be combined or distributed as desired in various embodiments.

Features of various aspects or embodiments have been described above by way of example, so that those skilled in the art may better understand the present invention. However, it is clear that aspects and embodiments other than those described in detail may also be subject matter of the invention, wherein the invention is defined by the scope of the appended claims.

List of reference signs

| | |
|---|---|
| 1 | skull |
| 2 | brain |
| 3 | probes |
| 4 | computer |
| 5 | computer-readable medium |

1. A computer-implemented method for classifying a brain pressure, the method comprising:
receiving data comprising time-of-flight values, which are based on time-of-flight values measured at successive time points for one or more ultrasound signals through a cross-section of a skull;
classifying a characteristic of the brain pressure by means of one or more features of a representative curve of a time course of the time-of-flight values to obtain a classified characteristic of the brain pressure; and
outputting the classified characteristic of the brain pressure.

2. The method according to claim 1, wherein the time course of the time-of-flight values correlates to a time course of the brain pressure within the skull.

3. The method according to claim 1, wherein the time course of the time-of-flight values comprises a plurality of peaks, which each correlate to an increase and a decrease of the brain pressure during a systolic and a diastolic phase.

4. The method according to claim 1, wherein a change between time-of-flight values in the time course at at least two successive time points correlates directly to a change of the brain pressure at the at least two successive time points.

5. The method according to claim 1, wherein the classified characteristic of the brain pressure indicates a normal brain pressure or indicates an abnormal brain pressure.

6. The method according to claim 1, wherein the representative curve comprises at least one characteristic peak.

7. The method according to claim 6, wherein the characteristic of the brain pressure is determined by the computer by extracting the one or more features of the curve of the at least one characteristic peak.

8. The method according to claim 7, wherein a first characteristic feature of the one or more features is a peak width of the at least one characteristic peak at a certain percentage of the maximum amplitude of the at least one characteristic peak.

9. The method according to claim 7, wherein a second characteristic feature of the one or more features is a similarity of the curve shape of the at least one characteristic peak to a synthetic function.

10. The method according to claim 6, wherein the at least one characteristic peak is characteristic for a plurality of peaks in at least one time segment of the time course of the time-of-flight values.

11. The method according to claim 10, wherein the at least one characteristic peak is determined by the following steps:
extracting a plurality of individual peaks from the at least one time segment of the time course of the time-of-flight values,
normalizing time points of the time-of-flight values in the individual peaks,
grouping the individual peaks by applying a similarity criterion for the individual peaks,
identifying the group with the largest number of peaks, and
forming the at least one characteristic peak from the peaks in the identified group of peaks.

12. The method according to claim 11, wherein forming the at least one characteristic peak comprises: for each time point, forming a median of the time-of-flight values of the peaks from the identified group.

13. The method according to claim 10, wherein the at least one segment is determined by the following steps:
extracting one or more time segments from the time course of the time-of-flight values, wherein a segment comprises a plurality of peaks in the time-of-flight values,
determining the at least one time segment by selecting one or more time segments in which the peaks meet a quality criterion.

14. The method according to claim 13, wherein the quality criterion is formed by an autocorrelation function which evaluates a self-similarity of the time course of the time-of-flight values in a segment.

15. The method according to claim 13, wherein the quality criterion is formed by a comparison of the maximum amplitudes of the most dominant frequency component with amplitudes of further frequency components in the time course of the time-of-flight values in a segment, or
wherein the quality criterion is formed by an evaluation of frequencies in the time course of the time-of-flight values in a segment.

16. (canceled)

17. The method according to claim 1, wherein receiving data with time-of-flight values comprises:
receiving a number of n time-of-flight curves, wherein in each of the n time-of-flight curves a time-of-flight value is assigned to one of the successive time points,
for each of the n time-of-flight curves, subtracting an offset from the time-of-flight values in the time-of-flight curve,
forming an averaged time-of-flight curve by averaging the time-of-flight values of each of the n time-of-flight curves at one of the successive time points.

18-21. (canceled)

22. A computer configured to carry out a method for classifying a brain pressure the method comprising:
receiving data comprising time-of-flight values, which are based on time-of-flight values measured at successive time points for one or more ultrasound signals through a cross-section of a skull;
classifying a characteristic of the brain pressure by means of one or more features of a representative curve of a time course of the time-of-flight values to obtain a classified characteristic of the brain pressure; and
outputting the classified characteristic of the brain pressure.

23. An apparatus arrangement, comprising the computer according to claim 22 and at least two probes, for determining the time-of-flight values of the one or more ultrasound signals through a cross-section of a skull, wherein the at least two probes comprise at least one emitter for outputting the one or more ultrasound signals and at least one corresponding detector for detecting the output one or more ultrasound signals.

24. (canceled)

25. The apparatus arrangement according to claim 23, wherein the at least two probes are configured
to output a plurality of wave packets each having an n-periodic signal of constant frequency at respective one of the successive time points,
to detect time-of-flight values through the cross-section of the skull for each of the n-periods of the periodic signal at each of the plurality of wave packets.

26. (canceled)

27. A computer-readable medium, wherein the medium comprises instructions which, when the method is executed by a computer, cause the computer to carry out a method for classifying a brain pressure,
the method comprising:
receiving data comprising time-of-flight values, which are based on time-of-flight values measured at successive time points for one or more ultrasound signals through a cross-section of a skull;
classifying a characteristic of the brain pressure by means of one or more features of a representative curve of a time course of the time-of-flight values to obtain a classified characteristic of the brain pressure; and
outputting the classified characteristic of the brain pressure.
